(19) United States
(12) Patent Application Publication
(10) Pub. No.: US 2011/0137185 A1
(43) Pub. Date: Jun. 9, 2011

(54) Saddle Faced Small Animal Sensor Clip

(75) Inventors: Bernard F. Hete, Kittanning, PA (US); Eric W. Starr, Allison Park, PA (US)

(73) Assignee: STARR LIFE SCIENCES CORP., Oakmont, PA (US)

(21) Appl. No.: 12/785,421

(22) Filed: May 21, 2010

Related U.S. Application Data
(60) Provisional application No. 61/180,162, filed on May 21, 2009.

(51) Int. Cl. A61B 5/02; A61B 5/00

(52) U.S. Cl. 600/504; 600/300

(57) ABSTRACT

A noninvasive photoplethysmographic sensor platform for small animals provides a spring biased sensor clip, wherein at least one side of the sensor clip is provided with a saddle faced clip face member. The saddle shape of the clip face member may have a hinge end, toward the hinge of the clip, which is longer in the longitudinal direction of the clip and shorter in depth than the distal end. The shorter distal end side (measured longitudinally) of this saddle shape facilitates the ability to align the transmitted and received light with the bone, while the overall saddle shape of the facing provides a physical grip to capture enough tissue to prevent the clip from relocating over time while it is attached to the limb. The shorter hinge end side (depth-wise) also allows the clip to close when it is fully assembled. The edges of the saddle shaped clip are preferably rounded off to prevent the contusions of the tissue that may result from long-term contact. This saddle faced feature works well on only one side of the clip.

The present invention claims priority of U.S. Provisional Patent Application Ser. No. 61/180,161 entitled “Saddle Faced Small Animal Sensor Clip”.

### BACKGROUND OF THE INVENTION

1. Field of the Invention

The present invention relates to photoplethysmographic readings for animal research and more particularly, the present invention is directed to a noninvasive photoplethysmographic sensor clip designed for improving the sensor position to maximize the usable signal.

2. Background Information

Photoplethysmography

A photoplethysmograph is an optically obtained plethysmograph, which, generically, is a measurement of changes in volume within an organ whole body, usually resulting from fluctuations in the amount of blood or air that the organ contains. A photoplethysmograph is often obtained by using a pulse oximeter.

A conventional pulse oximeter monitors the perfusion of blood to the dermis and subcutaneous tissue. Pulse oximetry is a non invasive method for the monitoring of the oxygenation of a subject's arterial blood, generally a human or animal patient or an animal (or possibly human) research subject. The patient/research distinction is particularly important in animals where the data gathering is the primary focus, as opposed to care-giving, and where the physiologic data being obtained may, necessarily, be at extreme boundaries for the subject.

As a brief history of pulse oximetry, it has been reported the first 2-wavelength earlobe O₂ saturation meter with red and green filters (later switched to red and infrared filters) was developed in 1935, which arguably, was the first noninvasive device to measure O₂ saturation. Further, in 1949, a pressure capsule was added to squeeze blood out of the earlobe to obtain zero setting in an effort to obtain absolute O₂ saturation value when blood was readmitted to the appendage. The concept of this early design is similar to today's conventional pulse oximetry but suffered due to unstable photocells and light sources and the method was not used clinically. In 1964, the first absolute reading ear oximeter that used eight wavelengths of light was commercialized by Hewlett Packard, however its use was limited to pulmonary functions due to cost and size.

Effectively, modern pulse oximetry was developed in 1972, by Aoyagi at Nihon Kohden using the ratio of red to infrared light absorption of pulsating components at the measuring site, and this design was commercialized by BIOX/Ohmeda in 1981 and Nellcor, Inc. in 1983. Prior to the introduction of these commercial pulse oximeters, a patient's oxygenation was determined by an arterial blood gas, a single point measure which typically took a minimum of 20-30 minutes processing by a laboratory. It is worthy to note that in the absence of oxygenation, damage to the human brain starts in 5 minutes with brain death in a human beginning in another 10-15 minutes.

Prior to the wide adoption of pulse oximetry, studies in anesthesia journals estimated US patient mortality as a consequence of undetected hypoxemia at 2,000 to 10,000 deaths per year, with no known estimate of patient morbidity. Consequently it is easy to see how pulse oximetry quickly became a standard of care for human patients since about 1987.

Pulse oximetry has also been a critical research tool for obtaining associated physiologic parameters in humans and animals beginning soon after rapid pulse oximetry became practical.

Magnetic Resonance Imaging (MRI)

Magnetic resonance imaging (MRI), or Nuclear magnetic resonance imaging (NMRI), is primarily a medical imaging technique most commonly used in radiology to visualize the structure and function of the subject tissue. The MRI provides detailed images of the tissue in any plane. MRI provides much greater contrast between the different soft tissues of a body than computed tomography (CT) does, making it especially useful in neurological (brain), musculoskeletal, cardiovascular, and oncological (cancer) imaging. Unlike CT, MRI uses no ionizing radiation, but uses a powerful magnetic field to align the nuclear magnetization of, generally, hydrogen atoms in water in the tissue.

Radiofrequency fields are used to systematically alter the alignment of this magnetization, causing the hydrogen nuclei to produce a rotating magnetic field detectable by the scanner. This signal can be manipulated by additional magnetic fields to build up enough information to construct an image of the body.

MRI is a relatively new technology, which has been in use for little more than 30 years (at the time of filing this application, as compared with over 110 years for X-Ray radiography). The first MR Image was published in 1973 and the first study performed on a human took place in 1977. Magnetic resonance imaging was developed from knowledge gained in the study of nuclear magnetic resonance. In its early years the technique was referred to as nuclear magnetic resonance imaging (NMRI). However, as the word nuclear was associated in the public mind with radiation exposure, thus it is generally now referred to simply as MRI.

Some research groups have used clinical MR scanners for imaging small animal. However, it has been found to be difficult to achieve a reasonable spatial resolution at an acceptable signal-to-noise ratio with these “large bore” scanners. Specialized “small-bore” MRI scanners are available for high-resolution MRI of small animals. In the drug development fields a discussion of the development and application of small bore MRIs for imaging of small animals can be found in the Markus Rudin 2005 book entitled “Imaging in Drug Discovery and Early Clinical Trials” available at http://www.springer.com.

The studies conducted in small bore MRIs have proven very beneficial. As a representative example, the Science Daily, on Oct. 2, 2008, announced that a new magnetic resonance imaging procedure performed on a small bore MRI can detect very early breast cancer in mice, including ductal carcinoma in situ (DCIS), a precursor to invasive cancer. Some of the tumors detected were less than 300 microns in diameter, which were the smallest cancers ever detected at that time by MRI. In light of these possibilities a number of research facilities have small bore MRI systems for research.

Due to the operation of the MRI, sensors, such as pulse oximeters, need to be designed to operate in the extremes of the MRI environment. Generally this is the elimination of ferrous material and/or heavily shielding selected components. Pulse oximeters designed for use in the MRI environment typically utilize fiber optic cables to transmit the transmitted and received signals into and out of the bore (i.e. the magnetic field).

Small Animal Photoplethysmography

In conventional pulse oximetry, a sensor is placed on a thin part of the subject's anatomy, such as a human fingertip or earlobe, or in the case of a neonate, across a foot, and two wavelengths of light, generally red and infrared wavelengths, are passed from one side to the other. Changing absorbance of each of the two wavelengths is measured, allowing determination of the absorbances due to the pulsing artery alone, excluding venous blood, skin, bone, muscle, fat, etc. Based upon the ratio of changing absorbance of the red and infrared light caused by the difference in color between oxygen-bound (bright red) and oxygen unbound (dark red or blue, in severe cases) blood hemoglobin, a measure of oxygenation (the percent of hemoglobin molecules bound with oxygen molecules) can be made. The measured signals are also utilized to determine other physical parameters of the subjects, such as heart rate (pulse rate).

Starr Life Sciences, Inc. has utilized pulse oximetry measurements to calculate other physiologic parameters such as breath rate, pulse distension, and breath distention and others, which can be particularly useful in various research applications.

In addressing animal pulse oximetry, particularly for small rodents, one approach has been to modify existing human or neonate oximeters for use with rodents. This approach has proven impractical as the human based systems can only stretch so far and this approach has limited the use of such adapted oximeters. For example, these adapted human oximeters for animals have an upper limit of heart range of around 400 or 450 beats per minute which is insufficient to address mice that have a conventional heart rate of 400-800 beats per minute.

Starr Life Sciences has developed a small mammal oximeter, rather than an adapted human model, that has effective heart rate measurements up to and beyond 900 beats per minute, and this is commercially available under the Mouse Ox™ oximeter brand.

Regarding animal pulse oximetry, consideration must be made for the particular subject or range of subjects in the design of the pulse oximeter, for example the sensor must fit the desired subject (e.g., a medical pulse oximeter for an adult human finger simply will not adequately fit onto a mouse). Consequently there can be significant design considerations in developing a pulse oximeter for small mammals or for neonates or for adult humans.

Starr Life Sciences has developed pulse oximetry clips for use with animals such as small mice and rats, and these clips are applicable for the tail, neck, thighs, and head of the animal. It should be noted that the different intended clip application areas of the clip on the animal result in distinct advantages and disadvantages for the signal. Consequently, none of the existing small animal pulse oximeter solutions previously developed adequately address clip migration when the clip is applied to selected locations of small mammals while maximizing the signal transmission received in such devices on small mammals. It is an object of the present invention to address the deficiencies of the prior art discussed above and to do so in an efficient, cost effective manner.

### SUMMARY OF THE INVENTION

When making transmission oxygen saturation measurements on a mouse thigh with existing small animal clips, an existing spring-loaded clip is used to couple the light source and receiver to opposite sides of the appendage. The present inventors have deduced several difficulties associated with making this type of measurement. The first is that if the faces of the clip are flat (which is found in conventional prior art clips), the applicant's believe that the visco-elastic nature of the tissue underlying the skin can cause the flat faced clip to move over time under the load of the spring. Any such clip movement would likely cause a slow degradation of the quality of the light signals over the duration of a continuous measurement and typically cause an interruption in the usable data that is obtained.

The second problem is that in a system where the intensity of the input light is low, as is the case with magnetic resonance imaging (MRI) measurements using fiber optic cables, which inherently significantly attenuate the input and received light, position of the sensor is more critical to obtaining good signals for measurement. Under low input light, it can take a researcher significant time to find a clip placement location where sufficient received light intensity exists to make quality measurements. As a general concept, the applicants believe that the best signals are obtained when light shines through large arteries. Large arteries typically lie close to bone, an anatomical characteristic that is beneficial for physically protecting the arteries. Thus, a sensor light path in a small mammal is more likely to provide good photoplethysmographic signals if the light passes around/near the limb bone.

The various embodiments and examples of the present invention as presented herein are understood to be illustrative of the present invention and not restrictive thereof and are non-limiting with respect to the scope of the invention.

According to one non-limiting embodiment of the present invention, a noninvasive photoplethysmographic sensor platform for small animals provides a spring biased sensor clip, wherein at least one side of the sensor clip is provided with a saddle faced clip face member.

A “saddle” or “saddle shape” within the meaning of this specification is referencing the longitudinal shape of the clip face in which the clip face includes distal end laterally extending projections (or flanges or ridges) each having a generally concave face.

The saddle shape of the clip face member may have a hinge distal end, toward the hinge of the clip, which is longer in the longitudinal direction of the clip and shorter in depth height than the opposed distal end as measured from the sensor position.

The shorter opposed distal end side (measured longitudinally from the sensor position) of this saddle shape facilitates the ability to align the transmitted and received light with the bone, while the overall saddle shape of the facing provides a physical grip to capture enough tissue to prevent the clip from relocating over time while it is attached to the limb.

The shorter (depth-wise or height wise) hinge end side also allows the clip to close when it is fully assembled. The edges of the projections at the ends of the saddle shaped clip are preferably rounded off to prevent the contusions of the tissue that may result from long-term contact.

This saddle faced feature works well on only one side of the clip, although such a design could be easily conceived that would be located on both clip halves.

The saddle faced clip face member can also be integrated as a part of the clip either by being built into it, or by being adhered with adhesive or with a press fit.

A further aspect of the present invention provides an integral diffuser into the clip face member which is aligned with the transmission source on the clip, wherein the diffuser includes a substantially encapsulated diffuser material provided within a diffuser pocket formed in the clip face member.

These and other advantages of the present invention will be clarified in the description of the preferred embodiments taken together with the attached figures.

### BRIEF DESCRIPTION OF THE DRAWINGS

FIG. 1 is a schematic representation of an existing noninvasive photoplethysmographic sensor platform for small animals, namely rats and mice, also known as a tail clip, to which the saddle faced feature in accordance with one embodiment of the present invention can be attached or can replace;

FIG. 2 is a schematic representation of an existing noninvasive photoplethysmographic sensor platform for small animals, namely rats and mice, also known as a neck collar, to which the saddle faced feature in accordance with one embodiment of the present invention can be attached or can replace;

FIG. 3 is an enlarged schematic top perspective view of a saddle faced clip attachment in accordance with one embodiment of the present invention;

FIG. 4 is an enlarged schematic bottom perspective view of the saddle faced clip attachment of FIG. 3;

FIG. 5 is an elevation schematic side view of a saddle faced clip on a clip half in accordance with one embodiment of the present invention; and

FIG. 6 is a schematic perspective top view of the saddle faced clip half of FIG. 5.

### DESCRIPTION OF THE PREFERRED EMBODIMENTS

In summary, the present invention relates to a noninvasive photoplethysmographic sensor platform 10, namely a sensor clip, for small animals, such as rats and mice that are typically utilized in a laboratory environment. The platform generally is operable on a computer or controller 12 with a display such as a lap top. The controller 12 may also include signal processing elements such as an external signal processing box. Cables 14 extend from the controller 12 to sensors 16 held within the animal engaging end 20 of a clip. The clip includes gripping elements 22, a biasing spring 24 and a pivot connection 26. Photoplethysmographic measurements on laboratory animals have most often been accomplished on restrained and/or anesthetized animals. Further, in the pulse oximetry field there has been a lack of adequate photoplethysmographic sensors for small mice (and even small rats), until the advent of the Mouse Ox™ brand pulse oximeters by Starr Life Sciences.

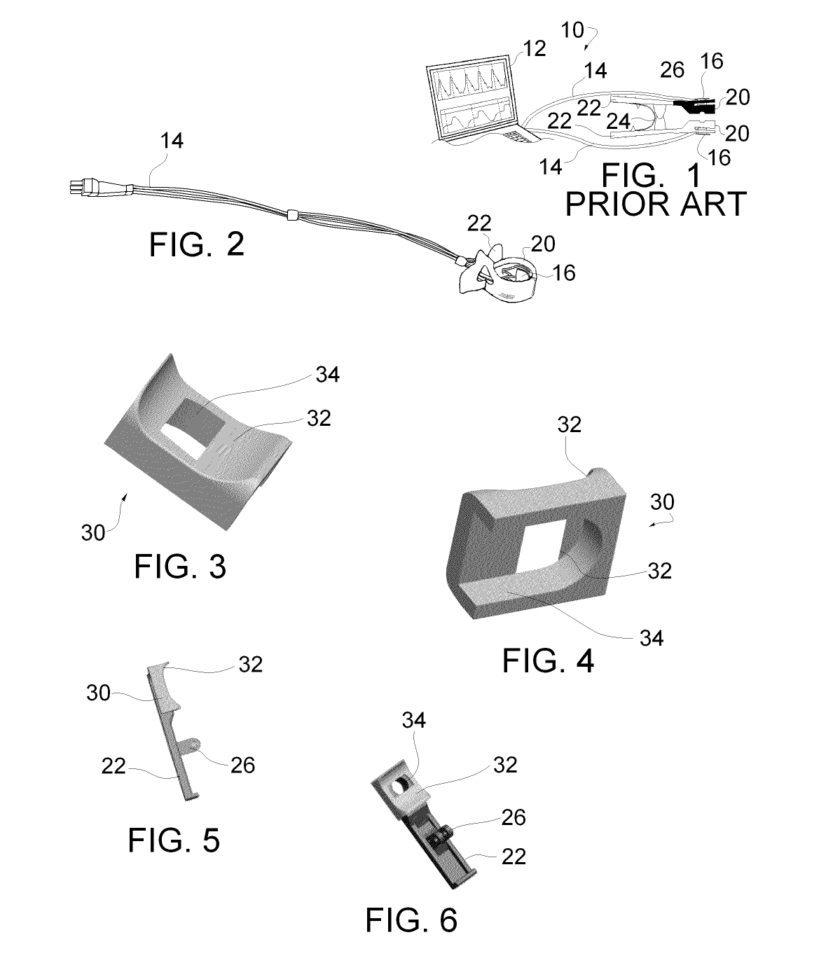

FIG. 1 is a schematic representation of a noninvasive photoplethysmographic sensor platform 10 for small rodents, namely rats and mice, to which the saddle faced clip face member 30 in accordance with one embodiment of the present invention can be applied as will be discussed below. A “saddle” or “saddle shape” within the meaning of this specification is referencing the longitudinal shape of the clip face 30 in which the clip face includes distal end laterally extending projections (or flanges or ridges) each having a generally concave face. The platform 10 is particularly well suited for use in a laboratory environment on a subject animal.

Within the meaning of this application the phrase “sensor clip” or term “clip” will reference a spring, or other closing biasing, member configured to receive sensor elements 16 thereon for application to the skin of the subject animal. The sensor elements 16 may be, for example, the transmitter and receiver of a pulse oximeter, or may be fiber optic cables, such as 14, that extend to such elements, such as in an MRI compatible version of a pulse oximeter.

The phrase “clip face member” within the meaning of this application will reference the tissue engaging end 30 of the clip, which may be integral with the clip or may be formed as separate attachable components.

The clip of FIG. 1 is a tail mounting clip designed by Starr Life Sciences. The tail provides certain advantages including ease of placement and, with the tail receiving groove in the end 20 of the clip, lack of significant movement of the clip. However, in certain applications the tail will not provide adequate measurements, and an alternative clip location must be selected. For example, when agitated or cold the mouse will shunt blood flow to the tail making measurements at this location unobtainable or more difficult. The saddle faced or saddle shaped clip face member 30 of the present invention, as shown and described herein, can be utilized with the tail clip of FIG. 1 to allow for more accurate repositioning of this clip to the thigh or other desired location.

The platform or clip will include a processor or controller 12 coupled thereto. The controller 12 is shown schematically in FIG. 1 and can be formed as a component of a laptop or desktop computer. The controller 12 may be the combination of stand alone hardware and software that is coupled with computer for the user interface, display memory and some computation. One particularly advantageous use of the photoplethysmographic measurements of the platform is for pulse oximetry, particularly in animals such as rats and mice. In this application the controller is the commercially available MouseOx™ product from Starr Life Sciences with the unique sensor mounting and coupling as described hereinafter. The details of the controller 12, including the user interface, the user display, memory or the like is not discussed herein in detail.

A conventional controller cable 14 extends from the controller for transmitting control and power signals from the controller and data back to the controller.

FIG. 2 illustrates includes sensors 16 mounted on a body encircling collar or neck clip, configured to encircle a subject animal body portion, such as, specifically around the neck of the subject animal. This neck clip was developed by Starr Life Sciences. The neck of small mammals such as rats and mice allows for a number of advantages for photoplethysmographic pulse oximetry measurements. The necks of animals of the sub-order muroidia tend to allow for both transmissive and reflective pulse oximetry measurements. Transmissive pulse oximetry is where the received light is light that has been transmitted through the perfuse tissue, whereas in reflective pulse oximetry the representative signal is obtained from light reflected back from the perfuse tissue. Each technique has its unique advantages. Transmissive techniques often result in a larger signal of interest, which is very helpful in small animals that have very small quantities of blood being measured to begin with. Reflective techniques can be used in environments that do not allow for transmissive procedures (e.g. the forehead of a human).

Further, the neck region of the animal offers an area with a relatively large blood flow for the animal, which will improve the accuracy of the measurements. In addition to increased blood flow, the blood flow is present under substantially all conditions. In other areas of the animal, such as the legs, paws and tail, the animal will often cut off blood flow under a variety of conditions. As discussed above, if the animal is cold or sufficiently agitated the blood flow to the tail can be shunted. The neck, in contrast represents an area of the animal that will always maintain a constant blood flow for measurements.

The neck collar also provides a bite proof location for the sensor mounting. In attempting to remove the sensors the biting of most animals, particularly animals of the sub-order muroidia, will be stronger than the clawing, and the neck location prevents the biting attacks as the animal cannot reach the collar. A secured collar cannot be removed by the animals paws or clawing.

Despite the described advantages of the neck location, there may be a need for repositioning of the clip to, for example, the thigh of the animal. One need would be to remove the clip from the area of interest in an MRI procedure. The saddle faced or saddle shaped clip face member 30 of the present invention, as shown and described herein, can be utilized with the tail clip of FIG. 2 to allow for more accurate repositioning of this clip to the thigh or other desired location.

As suggested above, the platform or clip of the present invention is not limited to sensors for photoplethysmographic measurements. Additional or alternative sensors can be used or added, such as temperature sensors and other physiologic and environmental sensors.

According to one non-limiting embodiment of the present invention, a noninvasive photoplethysmographic sensor platform for small animals provides a spring biased sensor clip, wherein at least one side of the sensor clip is provided with a saddle faced clip face member 30 which is shown in FIGS. 3-6. FIGS. 3-4 are enlarged schematic top and bottom perspective views of a saddle faced clip face member 30 as a separate attachment in accordance with one embodiment of the present invention. FIGS. 5-6 show a saddle faced clip face member 30 on a clip half in accordance with one embodiment of the present invention. The clip half of FIGS. 5-6 includes a pivot connection 26 and handles 22 and can receive a spring 24 for biasing the clip halves to a closed position.

The clip face includes laterally extending projections (or flanges or ridges) each having a generally concave face at each longitudinal distal end as shown. The shape of the clip face member has a hinge distal end, toward the hinge or pivot 26 of the clip, which is longer in the longitudinal direction of the clip and shorter in depth or height than the opposed distal end projection as measured from the sensor position at opening 34 or diffuser.

The shorter opposed distal end side projection (measured longitudinally from the sensor position) of this saddle shape facilitates the ability to align the transmitted and received light with the bone, while the overall saddle shape of the facing provides a physical grip to capture enough tissue to prevent the clip from relocating over time while it is attached to the limb.

The shorter hinge end side (depth-wise) also allows the clip to close when it is fully assembled. The edges of the saddle shaped clip are preferably rounded off to prevent the contusions of the tissue that may result from long-term contact.

This saddle faced feature works well on only one side of the clip, although such a design could be easily conceived that would be located on both clip halves.

The saddle faced clip face member 30 can also be integrated as a part of the clip either by being built into the clip or by being adhered with adhesive or with a press fit.

Further another aspect of the present invention includes boosting the light signal on the clip. One method of boosting the signal strength is to provide an integral diffuser into the clip face member 30 at the position of opening 34 which is aligned with the transmission (or the receiving) source on the clip, wherein the diffuser includes a substantially encapsulated diffuser material provided within a diffuser pocket formed in the clip face member.

The clip faced member 30 with integral diffuser may be formed of a first moldable material, e.g. plastic, that forms a substantially encapsulated diffuser pocket through with the transmitted (or possibly the received) light is passed. This structure can be formed through co-injection molding or on 3D printing machines. The encapsulation process allows semi-solids and non-solids to form the diffuser material. The use of such a diffuser can maximize the received signal that can be critical in certain applications such as in the MRI environment.

Although the present clip is particularly well suited for pulse oximeters as discussed above it can be used effectively for many sensors, essentially it could be used for any application in which a sensor is needed to clip onto the thigh of a small mammal subject. Temperature sensors, position sensors, blood pressure monitors are some examples.

Whereas particular embodiments of the invention have been described above for purposes of illustration, it will be evident to those skilled in the art that numerous variations of the details of the present invention may be made without departing from the spirit and scope of the present invention.

What is claimed is:

1. A noninvasive sensor platform for small animals includes sensor clip, wherein at least one side of the sensor clip is provided with a saddle faced clip member having a sensor coupled thereto and having laterally extending projections at distal ends of the clip member, wherein each laterally extending projection has a generally concave face.

2. The noninvasive sensor platform according to claim 1 wherein the sensor clip includes a pivot.

3. The noninvasive sensor platform according to claim 2 wherein saddle faced clip face member is provided only on only one side of the clip.

4. The noninvasive sensor platform according to claim 3 further including an integral diffuser into the clip face member which is aligned with one of the transmission or receiving source on the clip, wherein the diffuser includes a substantially encapsulated diffuser material provided within a diffuser pocket formed in the clip face member.

5. The noninvasive sensor platform according to claim 4 wherein the laterally extending projection at the pivot end of the clip face member has a shorter height than the laterally extending projection at the opposed end of the clip face member.

6. The noninvasive sensor platform according to claim 5 wherein the laterally extending projection at the pivot end of the clip face member has a longer longitudinal length from the sensor than the laterally extending projection at the opposed end of the clip face member.

7. The noninvasive sensor platform according to claim 2 further including an integral diffuser into the clip face member which is aligned with one of the transmission or receiving source on the clip, wherein the diffuser includes a substantially encapsulated diffuser material provided within a diffuser pocket formed in the clip face member.

8. The noninvasive sensor platform according to claim 7 wherein the laterally extending projection at the pivot end of the clip face member has a shorter height than the laterally extending projection at the opposed end of the clip face member.

9. The noninvasive sensor platform according to claim 8 wherein the laterally extending projection at the pivot end of the clip face member has a longer longitudinal length from the sensor than the laterally extending projection at the opposed end of the clip face member.

10. The noninvasive sensor platform according to claim 2 wherein the laterally extending projection at the pivot end of the clip face member has a shorter height than the laterally extending projection at the opposed end of the clip face member.

11. The noninvasive sensor platform according to claim 10 wherein the laterally extending projection at the pivot end of the clip face member has a longer longitudinal length from the sensor than the laterally extending projection at the opposed end of the clip face member.

12. The noninvasive sensor platform according to claim 2 wherein the laterally extending projection at the pivot end of the clip face member has a longer longitudinal length from the sensor than the laterally extending projection at the opposed end of the clip face member.

13. A noninvasive photoplethysmographic sensor platform for small animals includes a pivoted spring biased sensor clip, wherein at least one side of the sensor clip is provided with a saddle faced clip member having a photoplethysmographic sensor coupled thereto and having laterally extending projections at distal ends of the clip member, wherein each laterally extending projection has a generally concave face.

14. The noninvasive photoplethysmographic sensor platform according to claim 13 wherein saddle faced clip face member is provided only on only one side of the clip.

15. The noninvasive photoplethysmographic sensor platform according to claim 14 further including an integral diffuser into the clip face member which is aligned with one of the transmission or receiving source on the clip, wherein the diffuser includes a substantially encapsulated diffuser material provided within a diffuser pocket formed in the clip face member.

16. The noninvasive photoplethysmographic sensor platform according to claim 14 wherein the laterally extending projection at the pivot end of the clip face member has a shorter height than the laterally extending projection at the opposed end of the clip face member.

17. The noninvasive sensor photoplethysmographic platform according to claim 16 wherein the laterally extending projection at the pivot end of the clip face member has a longer longitudinal length from the photoplethysmographic sensor than the laterally extending projection at the opposed end of the clip face member.

18. The noninvasive photoplethysmographic sensor platform according to claim 13 further including an integral diffuser into the clip face member which is aligned with one of the transmission or receiving source on the clip, wherein the diffuser includes a substantially encapsulated diffuser material provided within a diffuser pocket formed in the clip face member.

19. The noninvasive photoplethysmographic sensor platform according to claim 13 wherein the laterally extending projection at the pivot end of the clip face member has a shorter height than the laterally extending projection at the opposed end of the clip face member.

20. The noninvasive photoplethysmographic sensor platform according to claim 13 wherein the laterally extending projection at the pivot end of the clip face member has a longer longitudinal length from the photoplethysmographic sensor than the laterally extending projection at the opposed end of the clip face member.
